(19) United States
(12) Patent Application Publication
(10) Pub. No.: US 2022/0062616 A1
(43) Pub. Date: Mar. 3, 2022

(54) TUBING JUNCTION ASSEMBLY

(71) Applicant: CareFusion 303, Inc., San Diego, CA (US)

(72) Inventor: Vincent Vinh NGUYEN, Placentia, CA (US)

(21) Appl. No.: 17/407,167

(22) Filed: Aug. 19, 2021

Related U.S. Application Data
(60) Provisional application No. 63/072,785, filed on Aug. 31, 2020.

(51) Int. Cl. A61M 39/12; A61M 39/10

(52) U.S. Cl. CPC A61M 39/12; A61M 2039/1038; A61M 39/1011

(57) ABSTRACT

Tubing junction assemblies that can be coupled with a portion of tubing, and can include a locking feature to resist unintended disassembly of the junction, and can include an assembly assistance feature to provide leverage when assembling a collar with a body of the tubing junction assembly, where the collar and body can be assembled together with a portion of tubing therebetween such that the tubing can be compressed between an inner surface of the collar and an outer surface of the body to fluidly couple the tubing with a bore through the body, provide a fluid-tight seal between the tubing and the body, and resist separation of the tubing from the body.

### CROSS-REFERENCE TO RELATED APPLICATIONS

This application claims the benefit of U.S. Provisional Patent Application No. 63/072,785 entitled “TUBING JUNCTION ASSEMBLY,” filed on Aug. 31, 2020, the disclosure of which is hereby incorporated by reference in its entirety for all purposes.

### BACKGROUND

The present disclosure relates generally to coupling tubing with another apparatus in a medical system, and more particularly, to fluidly coupling tubing with another apparatus in a medical system using a mechanical junction and without requiring adhesive, welding, or the similar bonding methods.

In the medical field, as well as in other fields and applications, tubing can be coupled with an apparatus or a fitting and used to transfer a fluid. In the medical field, patients can often be provided with medical fluids that are administered through intravenous (IV) infusion using assemblies of tubes and fittings commonly referred to as “IV sets.” IV sets are produced in a variety of configurations with various types of access ports, manifolds, valves, drip chambers, and other fittings connected by lengths of medical tubing.

The connections between the tubing and a fitting can be referred to as a “joint” or a “junction.” When the assembly of tubing and fitting are used in a medical application, or other applications requiring a high degree of integrity or performance, it is important to prevent the defects in the junction between the tubing and the fitting. It is also important to reduce the required steps for preparation and assembly of the junction during manufacturing.

A defect in the junction between the tubing and the fitting could result in a leakage passage between the fluid passage of the assembly and an environment outside of the fluid path. The leakage path may cause the medical fluid to exit the assembly or could permit air to enter the assembly, which could result in inadequate delivery of fluid and or medicament to the patient, the interaction of a gas the assembly, and/or could compromise the sterility of the assembly or medicament.

The junction can be achieved by bonding the tubing and the fitting together, such as by using a solvent, adhesive, welding, or the like. The junction can also be achieved by using a mechanical coupling, such as an interference fit it or a clamp. However, a variety of factors for coupling the tubing and fitting together must be considered to prevent the formation of a defect in the junction.

### SUMMARY

The junction between tubing and a fitting may fail to meet the integrity or performance requirements if the junction is defective. Some factors which may result in a defective junction include, the volume of solvent applied, the compatibility of the tubing and fitting materials with the solvent, the amount of time required or provided for curing the bond, the uniformity of the applied solvent, or the tolerance of the tubing and the fitting relative to each other. In some instances, manufacturing and assembly constraints do not permit the necessary time to carry out all necessary steps for coupling the tubing and junction, which may include checking and adjusting tolerances of the components relative to each other.

An aspect of the present disclosure provides a tubing junction assembly comprising a body having an outer surface, an inner surface, a proximal end portion, and a distal end portion, the inner surface defining a bore that extends between the proximal and distal end portions, and the outer surface having a body thread that extends from the outer surface in a direction that is radially outward and away from the bore, wherein the outer surface of the body defines a cross-sectional width that decreases from the proximal end portion toward the distal end portion of the body, and the tubing junction assembly comprises a collar having a first end, a second end, and an inner surface defining a passage that that extends between the first and second ends, the inner surface of the collar having a collar thread that extends radially inward into the passage, wherein the collar thread extends along the inner surface of the collar from the first end toward the second end of the collar.

The present disclosure provides tubing junction assembly comprising a body having an outer surface, an inner surface, a proximal end portion, a distal end portion, and a transverse wall between the proximal and distal end portions, the transverse wall comprises a cavity that extends through the wall toward the proximal end portion, and the tubing junction assembly comprises a collar having a first end, a second end, and an inner surface defining a passage that that extends between the first and second ends, wherein the collar comprises a locking tab that extends from the first end in a first direction away from the second end of the collar, wherein, when the collar is coupled with the body, the locking tab is configured to be inserted into the cavity of the body to resist movement of the collar away from the body.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a tubing junction assembly incorporated into an IV set coupled to a patient through, in accordance with aspects of the present disclosure.

FIG. 2 illustrates a perspective view of a tubing junction assembly, in accordance with aspects of the present disclosure.

FIG. 3 illustrates an exploded perspective view of the tubing junction assembly of FIG. 2, in accordance with aspects of the present disclosure.

FIG. 4 illustrates a cross-sectional exploded view of the tubing junction assembly of FIG. 2, in accordance with aspects of the present disclosure.

FIG. 5 illustrates a cross-sectional view of the tubing junction assembly of FIG. 2, in accordance with aspects of the present disclosure.

FIG. 6 illustrates a perspective view of a tubing junction assembly, in accordance with aspects of the present disclosure.

FIG. 7 illustrates a perspective view of a tubing junction assembly and socket, in accordance with aspects of the present disclosure.

FIG. 8 illustrates a top plan view of the tubing junction assembly and socket of FIG. 8, in accordance with aspects of the present disclosure.

FIG. 9 illustrates a cross-sectional view of the tubing junction assembly and socket of FIG. 8, in accordance with aspects of the present disclosure

FIG. 10 illustrates a perspective view of a tubing junction assembly and socket, in accordance with aspects of the present disclosure.

FIG. 11 illustrates an exploded perspective view of the tubing junction assembly of FIG. 10, in accordance with aspects of the present disclosure.

FIG. 12 illustrates a cross-sectional exploded view of the tubing junction assembly of FIG. 11, in accordance with aspects of the present disclosure.

FIG. 13 illustrates a cross-sectional exploded view of the tubing junction assembly of FIG. 10, in accordance with aspects of the present disclosure.

FIG. 14 illustrates a cross-sectional view of the tubing junction assembly of FIG. 10, in accordance with aspects of the present disclosure

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present inventions may be disclosed or shown in the context of an apparatus in a medical system, such embodiments can be used in other fluid transfer applications (such as the movement of food, beverages, fuel, lubrication, etc.). Furthermore, various applications of such embodiments and modifications thereto, which may occur to those who are skilled in the art, are also encompassed by the general concepts described herein.

In accordance with some embodiments, the present application discloses various features and advantages of a tubing junction assembly. The tubing junction assembly can be used to reliably and easily couple an end portion of tubing to another apparatus or device and permit the movement of a fluid therebetween. Further, the tubing junction assembly can provide an effective and reliable coupling between tubing and an apparatus that is comparable to or exceeds other methods such as solvent bonding. The tubing junction assembly of the present disclosure also minimizes and/or eliminates factors that could reduce the reliability and performance of the junction, and reduces the complexity of assembling the junction.

For example, the tubing junction assembly of the present disclosure provides a junction between tubing and an apparatus without requiring a specific volume of adhesive or solvent to be applied to a specific length of the tubing. The tubing junction assembly also does not require chemical compatibility of the material for each of the adhesive or solvent, tubing, and the apparatus for the coupling to be effective and reliable. Additionally, the tubing junction assembly can achieve an effective and reliable coupling with a greater degree of tolerance between the tubing and the apparatus. The simple and effective features of the present disclosure provides a tubing junction assembly that can be easily adapted to automated manufacturing or assembly operations. In some embodiments, the tubing junction assembly provides features that resist unintended disassembly of the junction, such as one or more locking feature.

The tubing junction assembly of the present disclosure includes, but is not limited to, a body and a collar, where the collar and body can be coupled together to engage against a portion of tubing positioned between the body and collar. Although the body and the collar are described or illustrated in isolation, it should be understood that any of the body and/or collar can be configured as a portion of another device or apparatus. For example, the body can be a portion of an IV bag, a drip chamber, a check valve, a flow controller, a fluid filter, a pump, or another apparatus or device.

To fluidly couple an end portion of tubing to the body, the end of the tubing is inserted onto the body so that a distal portion of the body extends into the fluid passage of the tubing. The collar is then inserted onto the body and the end tubing so that the tubing is compressed between the body and the collar as the collar is coupled to the body.

In some embodiments of the present disclosure, the tubing junction assembly can include one or more locking feature and/or assembly assistance feature. The locking feature can include any of a self-locking thread, ratcheting teeth, a locking tab, or the like. A self-locking thread can provide an interference fit between the body and the collar, such that rotation that would otherwise unscrew or separate the collar and the body is resisted. A portion of the collar or body can comprise ratcheting teeth configured to engage against a ridge or pawl of the other of the collar or body, such that rotation that would otherwise unscrew or separate the collar and the body is resisted. A locking tab can extend from any of the body and collar and be configured to engage against a ledge of the other of the collar or body, such that when the collar and body are coupled together the locking tab engages against the ledge to resist separation of the collar and body and/or rotation of the collar relative to the body.

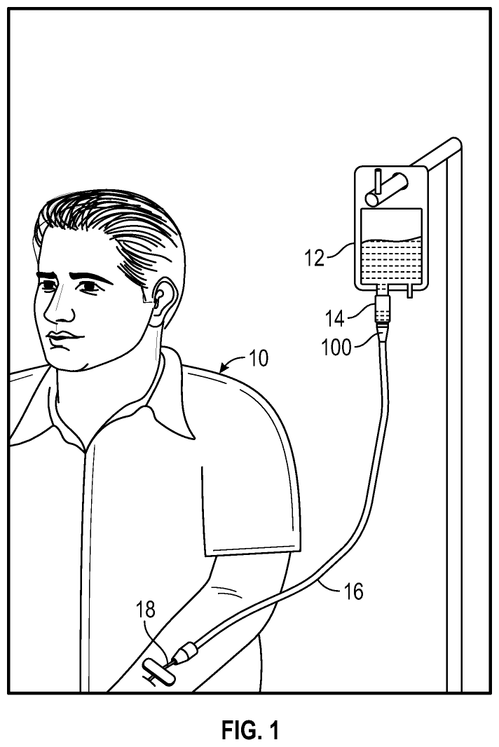

Referring now to the figures, FIG. 1 illustrates an example of a tubing junction assembly 100 in use in accordance with aspects of the present disclosure. The tubing junction assembly 100 is fluidly coupled with tubing of an intravenous (IV) set being used to deliver a fluid to a patient 10. The IV set includes a medicament bag 12, a drip chamber 14, tubing 16, the tubing junction assembly 100, and an IV catheter 18. Although the illustrated embodiment of the tubing junction assembly 100 is coupled with the drip chamber 14, it should be understood that the tubing junction assembly of the present disclosure can be incorporated into or coupled to other apparatus and devices, such as a manifold, a needleless access port, a check valve, a drip chamber, or other fluid transfer and/or control devices. Further, the tubing junction assembly of the present disclosure can be incorporated into or coupled to apparatus and devices used in other applications or industries other than medicine, including, for example, food, petroleum, nuclear utilities, transportation, mining, pulp and paper, steel, marine, aviation, construction, and other industries.

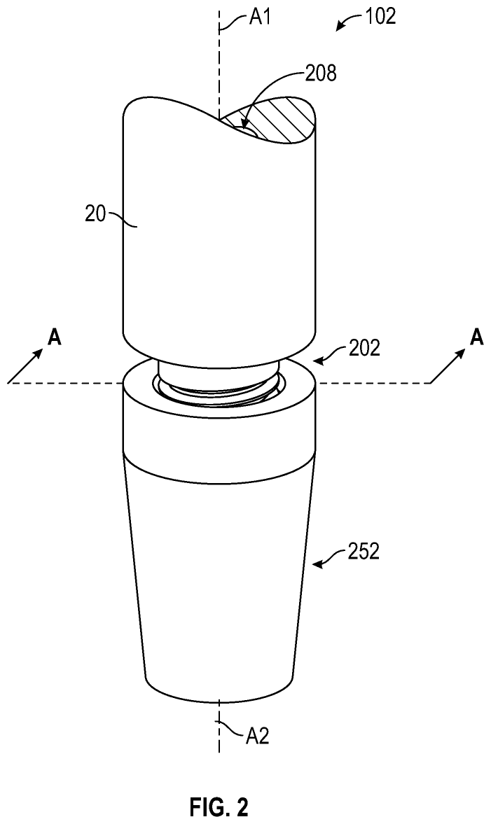

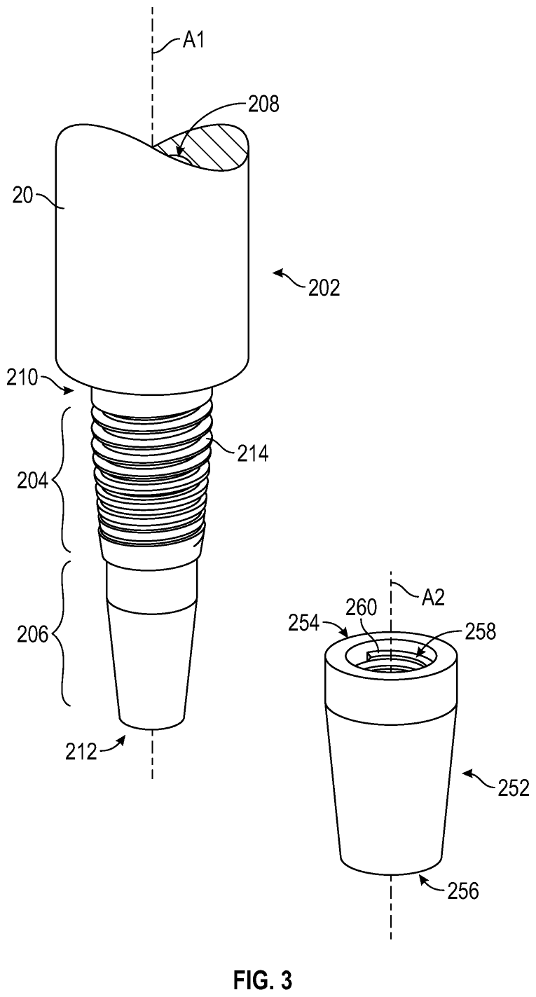

FIG. 2 is a perspective view of an embodiment of a tubing junction assembly 102 that is partially assembled. The tubing junction assembly 102 includes a body 202 and a collar 252. FIG. 3 is a perspective view of the tubing junction assembly 102 with the collar 252 separated from the body 202.

The tubing junction assembly 102 is configured to be assembled by moving the collar 252 onto a portion of the body 202. When an end portion of a tubing is positioned between the body 202 and the collar 252, the tubing is compressed between an outer surface of the body 202 and an inner surface of collar 252 as the collar is affixed to the body.

The body 202 includes a proximal end portion 204 and a distal end portion 206. A bore 208 extends through the proximal end portion 204 and the distal end portion 206, and is configured to direct a fluid through the body 202. The bore 208 defines an axis A1 the extends through the radial center of the bore 208, and between the proximal and distal end portions 204, 206 of the body. The distal end portion 206 of the body extends from the proximal end portion 202 to a distal end 212 of the body. The distal end portion 206 can be configured to be inserted into an end of a tubing so that at least a portion of the body 202 is positioned inside a bore of the tubing.

It is contemplated that the body 202 can be formed with another component 20 such as another apparatus or device. For example, a proximal end 210 of the body can be unitarily molded with another component 20. In another example, the body 202 can be coupled with another component 20 by applying an adhesive or welding the proximal end 210 of the body to the component 20. In yet another example, the body 202 can be coupled with another component 20 using a mechanical fastener, such as complementary threads, a latch, a clamp, or another of fastening the body to the component 20. As such, to simplify illustration of the features of the present disclosure, the body 202 is illustrated as being coupled with a portion of a component 20.

The collar 252 includes a first end 254 and a second end 256. A passage 258 extends through the first end 254 and the second end 256, and is configured to permit the collar to be inserted over at least a portion of the body 202 and the tubing. The passage 258 defines an axis A2 the extends through the radial center of the passage 258, and between the first and second ends 254, 256 of the collar.

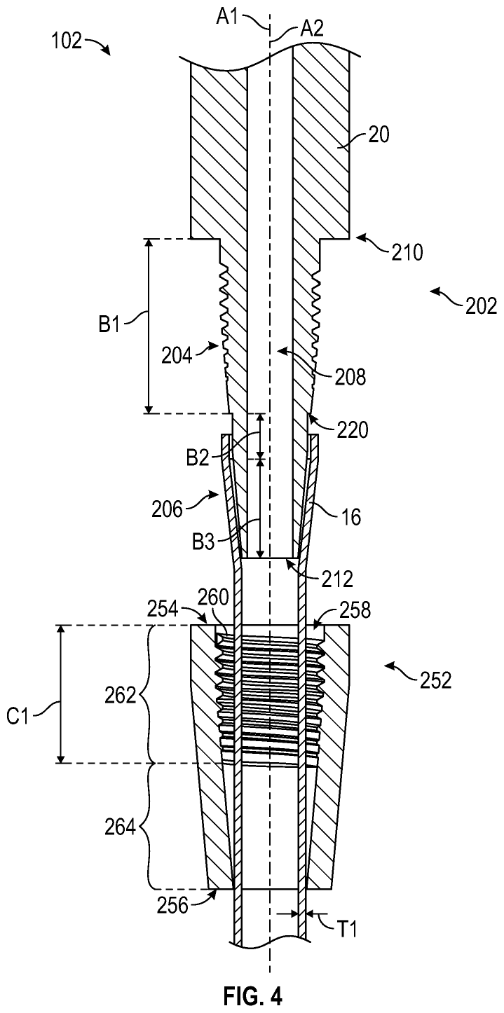

To facilitate coupling of the body 202 to the collar 252, the body 202 and the collar 252 can include complementary fasteners. The complementary fasteners include a body thread 214 that extends along the outer surface of the body 202 and a collar thread 260 that extends along an inner surface of the collar, as illustrated in FIGS. 3 and 4.

The body thread 214 can extend along any of the proximal end portion 204 and/or the distal end portion 206 of the body. As illustrated, the body thread 214 extends along the proximal end portion 204 of body, from a first point adjacent to the proximal end 210 of the body to a second point adjacent to the distal end portion 206 of the body. The body thread 214 can extend along a length B1 of the body, from the proximal end 210 partially toward the distal end 212 of the body.

The outermost surface of the body thread 214 forms an apex or ridge that defines an outer surface of the body. The outer surface of the body can have a cross-sectional width that decreases from the proximal end 210 toward the distal end portion 206 of the body. In some embodiments of the present disclosure, the cross-sectional width of the body can increase, decrease, and/or remain constant along proximal portion 204 of the body.

The distal end portion 206 of the body includes an outer surface that defines a cross-sectional width thereof. The cross-sectional width can be constant along a first segment of the distal end portion 206 having a length B2, and can decrease along a second segment of the distal end portion 206 having a length B3.

The cross-sectional width of the outer surface of the distal end 212 of the body can be approximately equal to a diameter of the bore of the tubing 16 configured to be coupled with the tubing junction assembly 102. When the distal end 206 of the body is inserted into the bore of the tubing 16, and the end of the tubing is moved along the outer surface of the body toward proximal end 210, an interference fit is established between the body and the tubing 16. In some embodiments of the present disclosure, the tubing 16 is stretched or swaged onto the body 202.

The body 202 can include a wall 220 configured to resist movement of the tubing 16, relative to the body 202, beyond the wall 220. The wall 220 can be formed by a portion of the outer surface of the body between the proximal and distal end portions 204, 206. The wall 220 extends in a direction that is transverse relative to the axis A1 through the bore 208. In some embodiments, the wall 220 can be formed as another structure, such as a ridge, post, or ramp that extends from the outer surface of the body 202. When the tubing 16 is coupled with the body 202, an end of the tubing 16 can engage against the wall 220 to resist movement of the tubing toward the proximal end 210 of the body 202.

To resist movement of the tubing 16 relative to the body 202, and to form a fluid-tight coupling between the tubing 16 and the body 202, the collar 252 can be coupled to the body 202. The collar 252 is configured to be coupled to the body 202 by inserting the collar 252 over the tubing 16 and onto at least a portion of the body 202.

To permit the collar 252 to be inserted over the body 202 and the tubing 16, the inner surface of the collar 252 that forms the passage 258 defines a cross-sectional width that is configured to allow the tubing and at least a portion of the body 202 to be inserted therethrough. The passage 259 can have a cross-sectional width that is approximately equal to a cross-sectional width of the body at an opposite point when the collar 252 is coupled to the body 202. In some embodiments, the cross-sectional width of the passage 258 decreases from the first end 254 toward the second end 256 of the collar.

The collar thread 260 extends along the inner surface of the collar, from the first end 254 toward the second end 256. The collar thread 260 extends along a length Cl of the collar defining a first end portion 262 from the first end 254 toward the second end 256 of the collar. In some embodiments, the length Cl of the collar is equal to the length B1 of the body. The passage 258 of collar also includes a second end portion 264 extending from the first end portion 262 to the second end 256.

In some embodiments, the complementary threads can extend along other portions of the body 202 and/or the collar 252. For example, a body thread can extend along an inner surface of the body 202 such as a sleeve of the body, and a collar thread can extends along an outer surface of the collar 252. It is also contemplated that the body 202 and collar 252 can include other forms of complementary fasteners, such as a cam lock, a latch, a barb, and/or an interference fit between opposing surfaces.

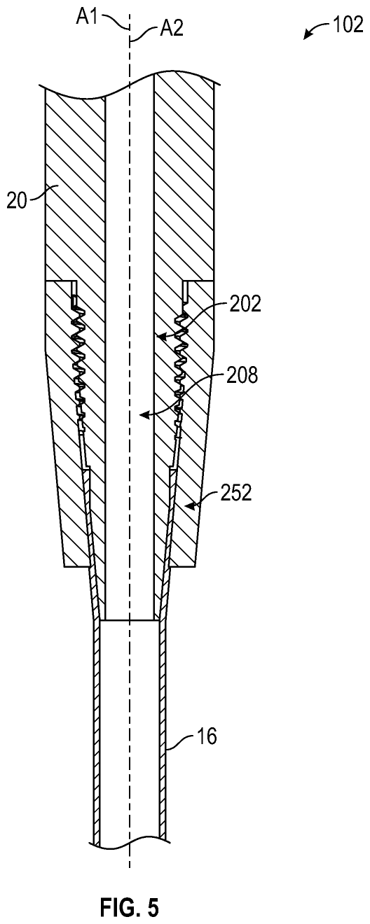

The tubing junction assembly 102 can be assembled with tubing 16 as shown in FIGS. 4 and 5, which illustrate cross-sectional views of the tubing junction assembly 102 through the line A-A of FIG. 2. To assemble the tubing junction assembly 102, the tubing 16 is inserted through the passage 258 of the collar. The distal end 212 of the body is inserted into the bore of the tubing 16 until at least some of the distal end portion 206 of the body is inserted into the tubing 16.

The collar 252 is then moved along the outer surface of the tubing 16 in a direction from the distal end 212 of the body toward the proximal end 210 of the body 202. In embodiments in which the body 202 and collar 252 have complementary threads, the collar 252 and/or body 202 can be rotated relative to each other.

When the collar 252 is coupled with the body 202, the distal end portion 206 of the body extends into the passage 258 of the collar. Additionally, a portion of, or all of, the proximal end portion 204 of the body can extend into the passage 258 of the collar. Further, when the collar 252 is coupled with the body 202, at least a portion of the outer surface of the distal end portion 206 of the body is spaced apart from the inner surface of the collar directly opposite to the body by a distance.

The distance between the collar 252 and the body 202 can be approximately equal to or less than a thickness T1 of the wall of the tubing 16. In some embodiments, the distance between the outer surface of the distal end portion 206 of the body and the inner surface of the collar, directly opposite to the body, is less than or equal to the thickness T1 of the tubing 16 such that the inner surface of the collar 202 engages against and compresses the tubing 16.

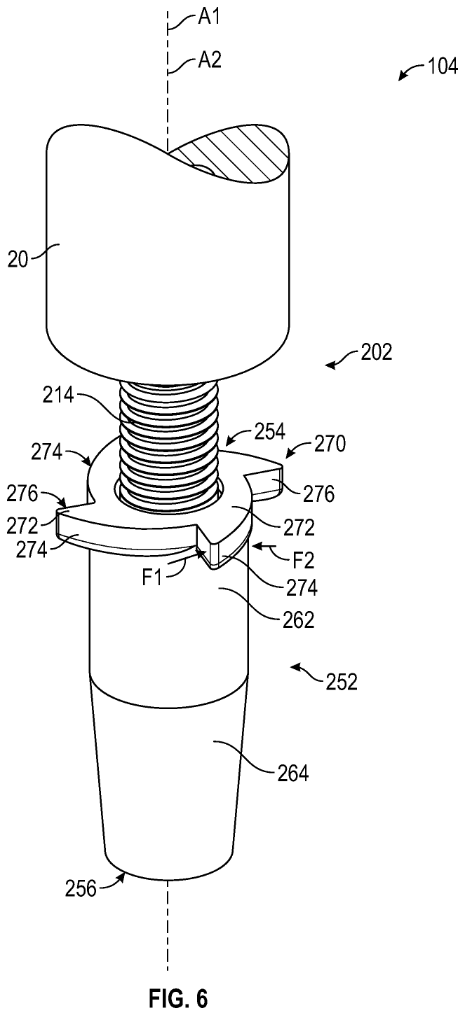

In some embodiments of the present disclosure the tubing junction assembly can include a feature to assist assembly the collar and the body. FIGS. 6-9 illustrate a tubing junction assembly 104 having an assembly assistance feature defined by a flange 270 that extends from the collar 252. The tubing junction assembly 104 can also include a tool configured to engage with the assembly assistance feature to facilitate assembly of the collar and the body. At least some features of the tubing junction assembly 104 are similar to the tubing junction assembly 102. Accordingly, for clarity and brevity of the present disclosure, reference numbers for features of the tubing junction assembly 104, which are the same or similar to features described relative to other embodiments of tubing junction assemblies of the present disclosure are repeated. It should be understood that features of the present disclosure can be combined or applied to embodiments of the tubing junction assemblies described in other portions of the present disclosure.

The flange 270 extends from an outer surface of the collar 252 in a direction that is radially outward and away from the collar 252 and transverse relative to the axis A2 of the collar.

The flange 270 can extend from the collar 252 along any of the first and second end portions 262, 264 of the collar. As illustrated, the flange 270 can extend from the outer surface of the collar at the first end 254 of the collar.

The flange 270 can include engagements surfaces that define one or more vertical plane parallel to the axis A2. The engagement surface can be defined by a spur 272 of the flange that extends radially outward and away from the collar 252. The flange 270 can include a plurality of spurs 272 spaced apart around a perimeter of the outer surface of the flange 270.

Each spur 272 forms a ramped surface 274 that extends away from the outer surface of the collar, and a transverse wall 276 that extends between the ramped surface 274 and the outer surface of the collar 252. In some embodiments of the present disclosure, each spur comprises an apex, where the ramped surface 274 extends from the outer surface of the collar 252 to the apex, and the transverse wall 276 extends from the apex to the outer surface of the collar 252.

At least one of the engagement surfaces is formed by the transverse wall 276 and defines a plane that intersects the axis A2. The transverse wall 276 is configured so that a force applied in the direction F1 against the transverse wall 276 causes the collar to rotate in a clockwise direction around the axis A2.

In contrast, a plane defined by the ramped surface 274 does not intersect the axis A2. As such, a force applied in the direction F2 against the ramped surface 274 is deflected away from flange 270 thereby resisting rotation of the collar in the counterclockwise direction around the axis A2.

In some embodiments, the assembly assistance feature can be formed by another feature or structure of the collar 252. For example, an assembly assistance feature can be defined by a post, ridge, or ramp that extends outward from an outer surface of the collar 252. In some embodiments, the assembly assistance feature can be defined by a dimple or groove that extends into the outer surface of the collar 252.

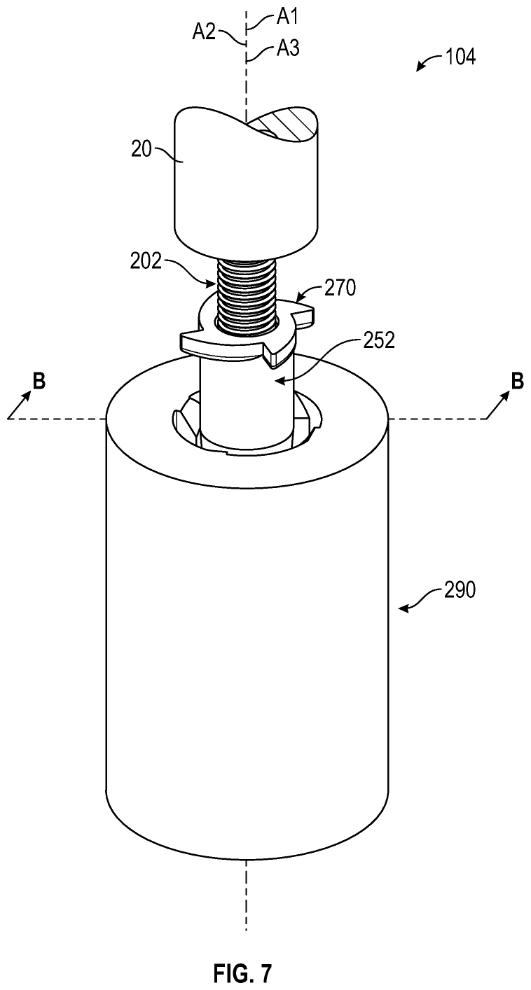

Referring to FIG. 7, the tubing junction assembly 104 can also include a socket 290 configured to mate with the flange 270 and facilitate coupling of the collar 252 with the body 202. The socket 290 can be configured to provide leverage to rotate the collar 252 when coupling the body and collar threads 214, 260 together. The socket 290 can also be used to align the collar 252 relative to the body 202 during assembly of the tubing junction assembly 104.

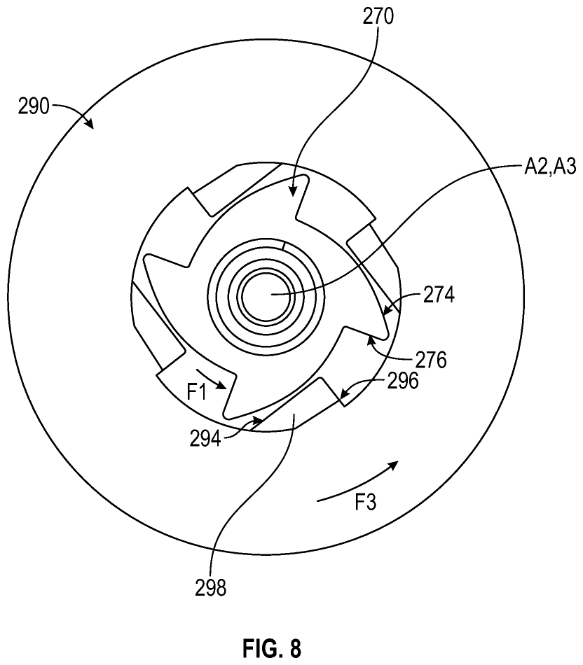

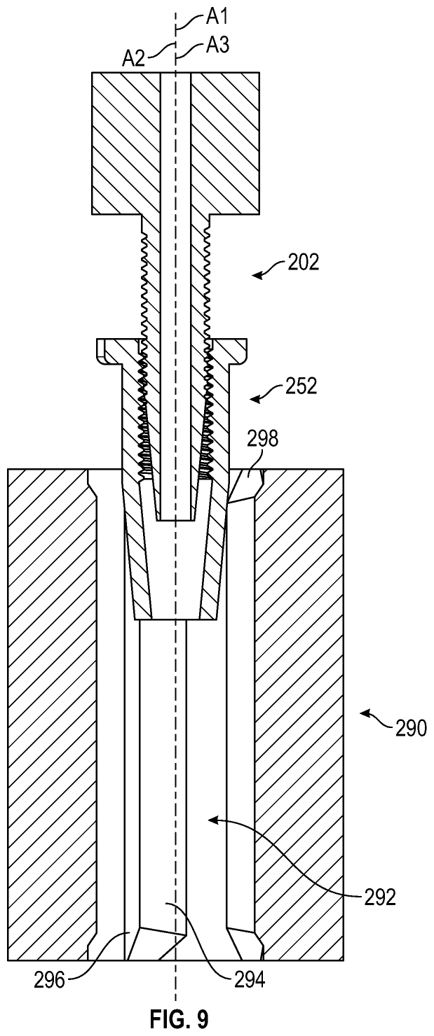

Referring to FIGS. 8 and 9, the socket comprises an inner surface defining a passage 292 having a socket axis A3. The inner surface forms a ramped surface 294 and a transverse wall 296 that extend into the passage 292. When the collar 252 is inserted into the socket 290, the transverse wall 296 of the socket is aligned with the transverse wall 276 of the flange, and the axis A2 of the collar and the axis A3 of the socket 290 are collinear. When the socket 290 is rotated in the first direction F3 around the socket axis A3, the transverse wall 296 of the socket engages against the transverse wall 276 of the flange, applying a force F1 thereon.

If the socket 290 is rotated in a second direction that is opposite to the direction F3, the ramped surface 294 of the socket engages against the ramped surface 274 of the flange such that the collar 252 is not rotated on the body 202. The collar 270 and the socket 290 are configured such that the transverse wall 296 of the socket does not engage against the transverse wall 276 of the flange when the socket 290 is rotated in the second direction, opposite to the direction F3.

The socket 290 can include a beveled surface 298 that extends from an end of the socket to an apex defined between the ramped surface 294 and the transverse wall 296. The beveled surface 298 is configured to engage against a portion of the spur 272 when the socket 290 is inserted into the collar 252 as shown in FIG. 9, which illustrates a cross-sectional view of the tubing junction assembly 104 through the line B-B of FIG. 7. When the beveled surface 298 engages against the spur 272, the collar 252 and/or the socket 290 are directed to rotate relative to each other until the transverse wall 296 of the socket is aligned with the transverse wall 276 of the flange.

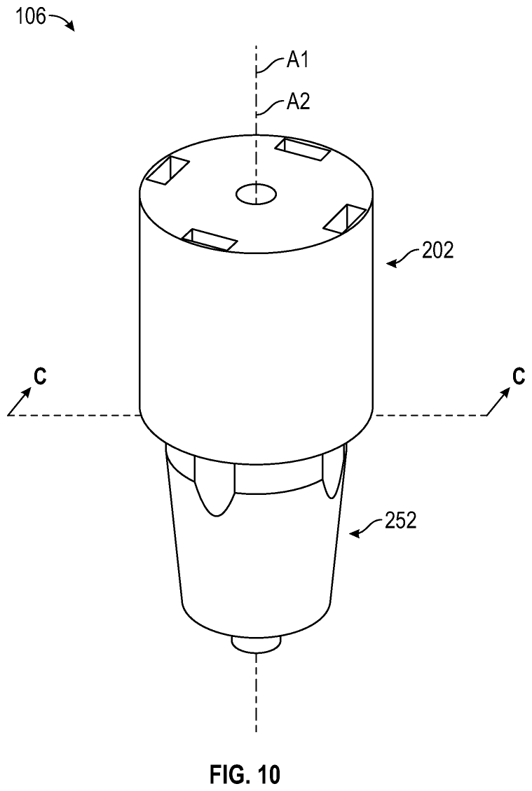

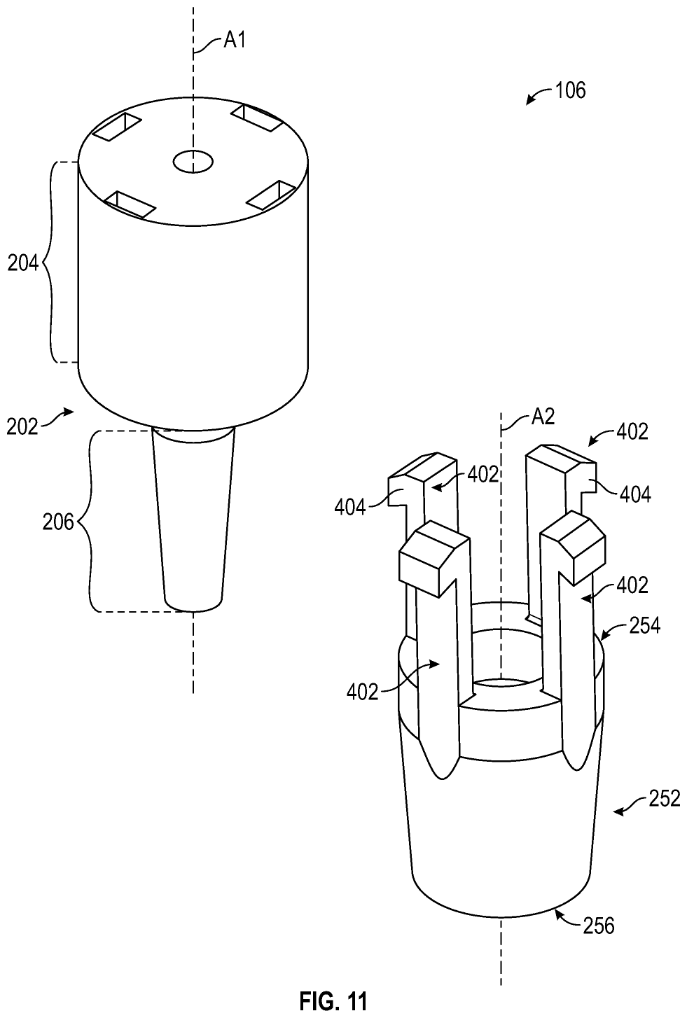

Referring to FIGS. 10-14, a tubing junction assembly 106 is illustrated having a locking feature. At least some features of the tubing junction assembly 106 are similar to the tubing junction assemblies 102, 104. Accordingly, as explained above, for clarity and brevity of the present disclosure, reference numbers for features of the tubing junction assembly 106, which are the same or similar to other embodiments of tubing junction assemblies 102, 104 are repeated.

The locking feature of the tubing junction assembly 106 is defined by the collar 252 having a locking tab 402 and the body 202 having a cavity 452 configured to receive the locking tab therein.

The collar 252 includes a first end 254 and a second end 256. A passage 258 extends through the first end 254 and the second end 256, and is configured to permit the collar to be inserted over the body 202. The passage 258 defines axis A2 that extends through the radial center of the passage 258, and between the first and second ends 254, 256 of the collar.

The collar 252 includes one or more locking tab 402 and is configured to engage against the body 202 when the collar 252 is coupled to the body 202. The locking tabs 402 extend from the first end 254 in a first direction away from the second end 256 of the collar. Each locking tab 402 can extend in a direction that is approximately parallel to the axis A2 of the collar. When the collar 252 is being coupled with the body 202, and the axis A2 of the collar is aligned with the axis A1 of the body, the locking tabs 402 extend toward the body 202.

In some embodiments, each locking tab 402 includes a barb 404 that extends from an outer surface of the locking tab 402. The barb 404 can have a distal end that extends from the locking tab 402 in a second direction that is transverse relative to the first direction in which the locking tab 402 extends. When the collar 252 is coupled to the body 202, the barb 404 is configured to engage against a portion of the body 202 to resist unintended movement of the collar 252 relative to the body 202. In some embodiments, the barb 404 is configured to resist movement of the collar 252 away from the body 202 along the axis A1 through the bore of the body.

The body 202 can include transverse wall 442 that extends from the body in a direction that is away from the axis A1 of the body. The transverse wall 442 can extend along the proximal end portion 204 of the body, from the proximal end 210 toward the distal end 212 of the body.

The transverse wall 442 has an inner surface that is spaced apart from the outer surface of the proximal portion 204 of the body to define a cavity 444 therebetween. The cavity 444 is configured to receive the locking tab 402 therein when the collar 252 is coupled to the body 202.

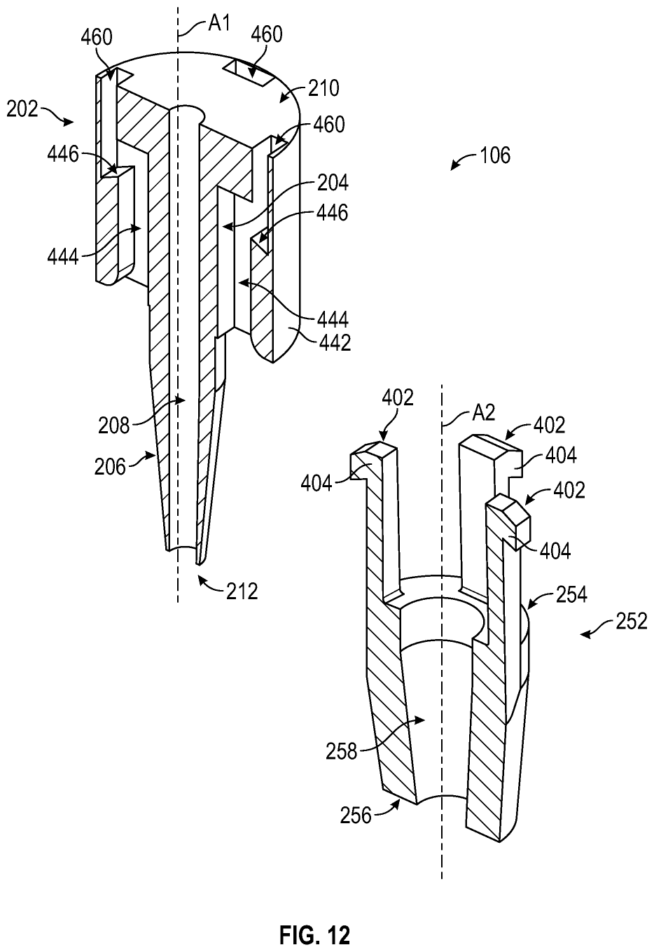

The body 202 can include a plurality of cavities 444 spaced apart around the axis A1 of the body and the outer surface of the proximal portion 204, as shown in FIGS. 12-14, which illustrate cross-sectional views of the tubing junction assembly 106 through the line C-C of FIG. 10. When the body 202 includes a plurality of cavities 444, the collar 252 can only be moved toward and coupled with the body 202 when each locking tab 402 is aligned with a complementary cavity 444. Further, engagement of the locking tabs 402 against transverse wall 442 can resist rotation of the collar 252 relative to the body 202 around the axes A1, A2.

In some embodiments of the present disclosure, the cavity 444 extends around the entire outer surface that defines the perimeter of the proximal portion 204 of the body, such that the locking tabs 402 can be inserted into the cavity 444 in any radial orientation relative to the body 202.

When a locking tab 402 is inserted into the cavity 444, movement of the body 202 and the collar 252 away from each other is resisted by engagement of the barb 404 against a ledge 446 of the body. The ledge 446 can extend from the transverse wall 442 into the cavity 444 and toward the bore 208 of the body.

When body 202 is inserted through the passage of 258 the collar 252, the barb 404 of each locking tab 402 engages against the inner surface of the transverse wall 442 to bias the locking tab 402 toward the axis A2 of the collar. When the collar 252 is moved further toward the proximal end 210 of the body, the barb 404 moves past the ledge 446 such that the locking tab 402 can move radially outward toward an unbiased position.

In some embodiments of the present disclosure, the ledge 446 can extend from the outer surface of the proximal portion 204 of the body into the cavity 444. In such an embodiment, a locking tab 402 can be biased radially outward and away from the axis A1 of the body until a barb 404 of the locking tab moves past the ledge 446.

The body 202 can also include an aperture 460 that extends through the proximal end 210 of the body to the cavity 444. Where the body 202 includes a plurality of cavities 444, each cavity can include an aperture 460 extending through the proximal end 210 of the body.

The aperture 460 can intersect the cavity 444 at a portion adjacent to the ledge 446, such that an opening of the aperture in the cavity 444 is opposite to the ledge 446. The aperture 460 can permit an object to be inserted through the aperture 460 to bias a locking tab 402 and permit separation of the collar 252 from the body 202. For example, the collar 252 can be separated from the body 202 by inserting an object such as a pin through the aperture 460 until the pin engages against the barb 404 and biases the locking tab 402 away from the ledge 446. Once the locking tab 402 is biased a distance that is equal to a length of the barb 404, the collar 252 can be moved away from the body 202 such that the locking tab 402 is withdrawn from the cavity 444 without the barb 404 engaging against the ledge 446.

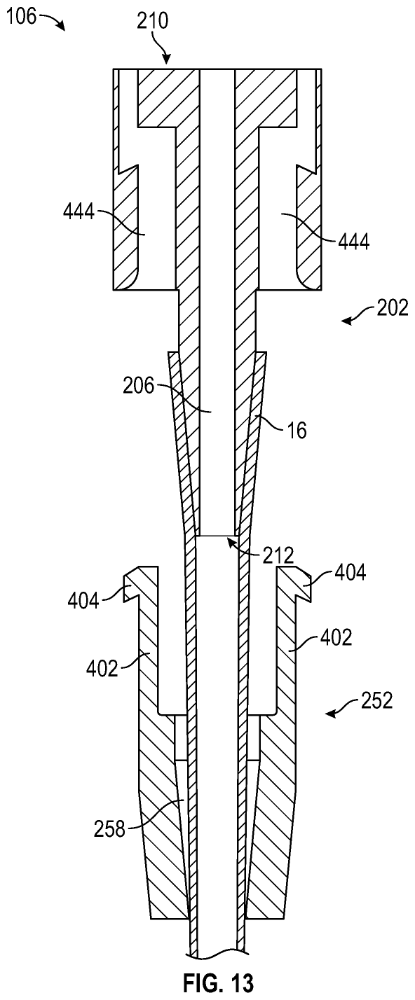

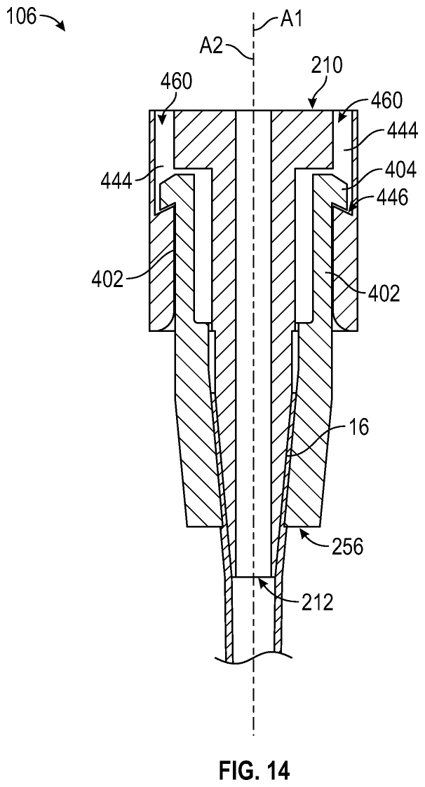

Referring to FIGS. 13 and 14, the tubing junction assembly 106 can be assembled by inserting the distal end portion 206 of the body into the bore of the tubing 16. The collar 252 is then moved along the outer surface of the tubing 16 in a direction toward the distal end 212 of the body.

In embodiments in which the body 202 has a plurality of cavities 444 spaced apart from each other, the body 202 and/or the collar 252 are rotated relative to each other until the locking tab 402 is aligned with a cavity 444. After the locking tab 402 and the cavity 444 are aligned, the collar 252 can be moved toward the proximal end 210 of the body such that the locking tab 402 enters into the cavity 444.

The collar 252 is moved in a first direction toward the proximal end 210 of the body 202 until the barb 404 of the locking tab is moved past the ledge 446. When the barb 404 is moved past the ledge, e.g. between the ledge 446 and the proximal end 210 of the body 202, the locking tab 402 is unbiased and moves away from the axis A2 of the collar 252 such that the barb 404 can engage against the ledge 446 when the collar is moved in a second direction away from the proximal end 210 of the body 202.

The tubing junction assembly 106 is configured such that, when the collar 252 is coupled with the body 202, the outer surface of the distal end portion 206 of the body is spaced apart from the inner surface of the collar directly opposite to the body by a distance. The distance between the distal end portion 206 of the body and the inner surface of the collar 252 is approximately equal to or less than a thickness T1 of the wall of the tubing 16. In some embodiments, the distance between the outer surface of the distal end portion 206 of the body and the inner surface of the collar, directly opposite to the body, is less than or equal to the thickness T1 of the tubing 16 such that the inner surface of the collar 202 engages against and compresses the tubing 16.

It should be understood that any of the features of the present disclosure can be included in any embodiment herein. For example, it is contemplated that a tubing junction assembly could be configured with a body and collar having corresponding mating threads, while also including a locking tab extending from one of the body and collar, the locking tab configured to engage the other of the body and collar when the body and collar are coupled together. In such an embodiment, engagement of the locking tab could provide an indication that the collar and body are fully coupled together as intended, and could resist decoupling of the collar from the body. Similarly, a tubing junction assembly of the present disclosure could include any of threaded coupling feature, an assembly assistance feature such as a flange, and locking feature.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A tubing junction assembly comprising: a body having an outer surface, an inner surface, a proximal end portion, and a distal end portion, the inner surface defining a bore that extends between the proximal and distal end portions, and the outer surface having a body thread that extends from the outer surface in a direction that is radially outward and away from the bore, wherein the outer surface of the body defines a cross-sectional width that decreases from the proximal end portion toward the distal end portion of the body; and a collar having a first end, a second end, and an inner surface defining a passage that that extends between the first and second ends, the inner surface of the collar having a collar thread that extends radially inward into the passage, wherein the collar thread extends along the inner surface of the collar from the first end toward the second end of the collar.

Clause 2. The tubing junction assembly of Clause 1, wherein the outer surface of the body comprises a wall between the proximal and distal end portions, wherein the wall extends in a direction that is transverse relative to an axis through the bore.

Clause 3. The tubing junction assembly of any of Clauses 1 or 2, wherein the body thread defines a cross-sectional width of the body that decreases from the proximal end portion toward the distal end portion.

Clause 4. The tubing junction assembly of any of Clauses 1 to 3, wherein the body thread extends along the proximal end portion.

Clause 5. The tubing junction assembly of any of Clauses 1 to 4, wherein the body thread extends along the proximal and distal end portions of the body.

Clause 6. The tubing junction assembly of any of Clauses 1 to 5, wherein the outer surface of the body along the distal end portion defines a cross-sectional width that is constant along a first distal portion length.

Clause 7. The tubing junction assembly of Clause 6, wherein the cross-sectional width of the outer surface of the body along the distal end portion decreases along a second distal portion length extending from the first distal portion length to a distal end of the body.

Clause 8. The tubing junction assembly of any of Clauses 1 to 7, wherein the passage comprises a length from the first end to the second end of the collar, and the collar thread extends along a portion of passage length.

Clause 9. The tubing junction assembly of any of Clauses 1 to 8, wherein the inner surface of the collar and the thread define a cross-sectional width of the passage that decreases from the first end toward the second end of the collar.

Clause 10. The tubing junction assembly of any of Clauses 1 to 9, wherein the collar thread extends along a first length, from the first end toward the second of the collar, and the body thread extends along a second length, wherein the first and second lengths are approximately equal.

Clause 11. The tubing junction assembly of any of Clauses 1 to 10, wherein, when the collar is coupled with the body, the distal end portion of the body extends into the passage of the collar.

Clause 12. The tubing junction assembly of Clause 11, wherein, when the collar is coupled with the body, the outer surface of the body along the distal end portion is spaced apart from the inner surface of the collar.

Clause 13. The tubing junction assembly of any of Clauses 1 to 12, wherein the collar comprises a flange that extends from the outer surface in a direction that is radially outward and away from the passage.

Clause 14. The tubing junction assembly of Clause 13, wherein the flange is positioned at the first end of the collar.

Clause 15. The tubing junction assembly of Clause 13, wherein the flange comprises a spur that extends radially outward and away from the passage.

Clause 16. The tubing junction assembly of Clause 15, wherein the spur comprises a ramped surface that extends away from the outer surface of the collar, and a transverse wall that extends between the ramped surface and the outer surface of the collar.

Clause 17. The tubing junction assembly of Clause 11, further comprising a socket having an inner surface defining a passage having a socket axis, wherein the inner surface comprises a ramped surface and a transverse wall, such that the transverse wall of the socket is configured to engage against the transverse wall of the collar flange when the socket is rotated about the socket axis in a first direction, and the transverse wall of the socket is configured to not engage against the transverse wall of the collar flange when the socket is rotated about the socket axis in a second direction which is opposite to the first direction.

Clause 18. The tubing junction assembly of Clause 17, wherein the socket comprises a beveled surface extending from a first end of the socket to a ridge between the ramped surface and the transverse wall.

Clause 19. A tubing junction assembly comprising: a body having an outer surface, an inner surface, a proximal end portion, a distal end portion, and a transverse wall between the proximal and distal end portions, the transverse wall comprises a cavity that extends through the wall toward the proximal end portion; and a collar having a first end, a second end, and an inner surface defining a passage that that extends between the first and second ends, wherein the collar comprises a locking tab that extends from the first end in a first direction away from the second end of the collar; wherein, when the collar is coupled with the body, the locking tab is configured to be inserted into the cavity of the body to resist movement of the collar away from the body.

Clause 20. The tubing junction assembly of Clause 19, wherein, when the collar is coupled with the body, the distal end portion of the body extends into the passage of the collar.

Clause 21. The tubing junction assembly of Clause 20, wherein, when the collar is coupled with the body, the outer surface of the body along the distal end portion is spaced apart from the inner surface of the collar.

Clause 22. The tubing junction assembly of any of Clauses 19 to 21, wherein the body comprises a ledge that extends into the cavity, and the locking tab comprises a barb, and wherein the barb is configured to engage against the ledge to resist movement of the collar away from the body along an axis through the bore when collar and body are coupled together.

Clause 23. The tubing junction assembly of Clause 22, wherein the ledge extends radially inward toward the bore axis, and the barb extends radially outward away from an axis through the passage.

Clause 24. The tubing junction assembly of Clause 22, wherein the barb comprises a distal end surface that extends in a second direction that is transverse relative to the first direction.

Clause 25. The tubing junction assembly of any of Clauses 19 to 24, wherein the body comprises a proximal end surface and an aperture that extend from the cavity through the proximal end surface.

Clause 26. The tubing junction assembly of any of Clauses 19 to 25, wherein the collar comprises a plurality of tabs spaced around a perimeter of the first end of the collar.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A tubing junction assembly comprising:
a body having an outer surface, an inner surface, a proximal end portion, and a distal end portion, the inner surface defining a bore that extends between the proximal and distal end portions, and the outer surface having a body thread that extends from the outer surface in a direction that is radially outward and away from the bore, wherein the outer surface of the body defines a cross-sectional width that decreases from the proximal end portion toward the distal end portion of the body; and
a collar having a first end, a second end, and an inner surface defining a passage that that extends between the first and second ends, the inner surface of the collar having a collar thread that extends radially inward into the passage, wherein the collar thread extends along the inner surface of the collar from the first end toward the second end of the collar.

2. The tubing junction assembly of claim 1, wherein the outer surface of the body comprises a wall between the proximal and distal end portions, wherein the wall extends in a direction that is transverse relative to an axis through the bore.

3. The tubing junction assembly of claim 1, wherein the body thread defines a cross-sectional width of the body that decreases from the proximal end portion toward the distal end portion.

4. The tubing junction assembly of claim 1, wherein the body thread extends along the proximal end portion.

5. The tubing junction assembly of claim 1, wherein the outer surface of the body along the distal end portion defines a cross-sectional width that is constant along a first distal portion length.

6. The tubing junction assembly of claim 5, wherein the cross-sectional width of the outer surface of the body along the distal end portion decreases along a second distal portion length extending from the first distal portion length to a distal end of the body.

7. The tubing junction assembly of claim 1, wherein the inner surface of the collar and the thread define a cross-sectional width of the passage that decreases from the first end toward the second end of the collar.

8. The tubing junction assembly of claim 1, wherein the collar thread extends along a first length, from the first end toward the second of the collar, and the body thread extends along a second length, wherein the first and second lengths are approximately equal.

9. The tubing junction assembly of claim 1, wherein, when the collar is coupled with the body, the distal end portion of the body extends into the passage of the collar.

10. The tubing junction assembly of claim 9, wherein, when the collar is coupled with the body, the outer surface of the body along the distal end portion is spaced apart from the inner surface of the collar.

11. The tubing junction assembly of claim 1, wherein the collar comprises a flange that extends from the outer surface in a direction that is radially outward and away from the passage.

12. The tubing junction assembly of claim 11, wherein the flange comprises a spur that extends radially outward and away from the passage.

13. The tubing junction assembly of claim 12, wherein the spur comprises a ramped surface that extends away from the outer surface of the collar, and a transverse wall that extends between the ramped surface and the outer surface of the collar.

14. The tubing junction assembly of claim 11, further comprising a socket having an inner surface defining a passage having a socket axis, wherein the inner surface comprises a ramped surface and a transverse wall, such that the transverse wall of the socket is configured to engage against the transverse wall of the collar flange when the socket is rotated about the socket axis in a first direction, and the transverse wall of the socket is configured to not engage against the transverse wall of the collar flange when the socket is rotated about the socket axis in a second direction which is opposite to the first direction.

15. The tubing junction assembly of claim 14, wherein the socket comprises a beveled surface extending from a first end of the socket to a ridge between the ramped surface and the transverse wall.

16. A tubing junction assembly comprising:
a body having an outer surface, an inner surface, a proximal end portion, a distal end portion, and a transverse wall between the proximal and distal end portions, the transverse wall comprises a cavity that extends through the wall toward the proximal end portion; and
a collar having a first end, a second end, and an inner surface defining a passage that that extends between the first and second ends, wherein the collar comprises a locking tab that extends from the first end in a first direction away from the second end of the collar;
wherein, when the collar is coupled with the body, the locking tab is configured to be inserted into the cavity of the body to resist movement of the collar away from the body.

17. The tubing junction assembly of claim 16, wherein the body comprises a ledge that extends into the cavity, and the locking tab comprises a barb, and wherein the barb is configured to engage against the ledge to resist movement of the collar away from the body along an axis through a bore of the body when the collar and the body are coupled together.

18. The tubing junction assembly of claim 17, wherein the ledge extends radially inward toward the bore axis, and the barb extends radially outward away from an axis through the passage.

19. The tubing junction assembly of claim 17, wherein the barb comprises a distal end surface that extends in a second direction that is transverse relative to the first direction.

20. The tubing junction assembly of claim 16, wherein the collar comprises a plurality of tabs spaced around a perimeter of the first end of the collar.
